(19) United States
(12) Patent Application Publication
(10) Pub. No.: US 2017/0188926 A1
(43) Pub. Date: Jul. 6, 2017

(54) EVALUATION DEVICE FOR DEGREE OF CONCENTRATION AND PROGRAM

(71) Applicant: Panasonic Intellectual Property Management Co., Ltd., Osaka (JP)

(72) Inventor: Fumiaki OOBAYASHI, Osaka (JP)

(21) Appl. No.: 15/313,832

(22) PCT Filed: Apr. 15, 2015
(86) PCT No.: PCT/JP2015/002078

§ 371 Date: Nov. 23, 2016

(30) Foreign Application Priority Data
May 26, 2014 (JP) 2014-108359

(51) Int. Cl. A61B 5/16

(52) U.S. Cl. CPC A61B 5/16

(57) ABSTRACT

An evaluation device that works out an objective evaluation value of a degree of concentration is provided. The evaluation device includes an acquirer, a storage, a processor and a corrector. The acquirer is configured to acquire a processing time measured for each of tasks performed by a user. The storage is configured to store the processing time for each of the tasks. The corrector is configured to, based on load factors that are set in accordance with respective degrees of difficulty of the tasks, correct and change the processing times stored in the storage to respective effective times corresponding to a prescribed degree of difficulty. The processor is configured to work out an evaluation value of user's degree of concentration based on a statistic acquired from a collection of the effective times.

### TECHNICAL FIELD

The invention relates generally to evaluation devices for degree of concentration and programs and, more particularly, to an evaluation device for degree of concentration configured to evaluate degree of concentration of a worker who performs mental work and a program for allowing a computer to function as main part of the evaluation device for degree of concentration.

### BACKGROUND ART

In a related technology for measuring user's cognitive capability, it has been known to calculate a capable score based on a time (a perception time) from a time at which a degraded image containing a meaningful subject is shown to the user to a time at which the subject is perceived by the user (see, for example Document 1 “JP 2006-087743 A”). Document 1 describes a technology which works out information on a degree of difficulty of the image to be perceived by showing two or more users the image in advance, and which calculates a capable score of a particular user based on a perception time obtained by showing the user the image, and the information on the degree of difficulty of the image.

In a related technology, it has been proposed to make a user trace a reference diagram to work out a degree of concentration from a difference amount between the reference diagram and the traced diagram (see, for example Document 2 “JP H09-135826 A”). The degree of concentration is worked out as a value obtained by multiplying a coefficient and a value obtained from the difference amount between the reference diagram and the traced diagram. At the end of the tracing operation, it calculates a change, a mean value, a standard deviation, a coefficient of variation, maximum and minimum values and the like about degrees of concentration worked out at regular intervals during the tracing operation. Document 2 further describes varying a degree of difficulty by changing a speed of the tracing operation when the reference diagram is traced, and estimating user's physiological state or character through the tracing operation of the reference diagram.

The capable score described in Document 1 just represents the capability for perceiving respective meaning of individual images, and it is not possible to evaluate the degree of concentration by the capable score when a mental work load is given to the user.

On the other hand, in the case of the technology described in Document 2, it is possible to work out a change in user's degree of concentration while a question as the tracing operation is provided, but it is however unsuitable for the evaluation of the degree of concentration because the tracing operation is not a mental work load. In addition, the tracing operation depends on exercise capacity of user's handwork and therefore the technology is not suitable for evaluating the degree of concentration from this point of view.

### SUMMARY OF INVENTION

It is an object of the present invention to provide an evaluation device for degree of concentration, capable of working out an objective evaluation value of a degree of concentration. It is also an object of the present invention to provide a program for allowing a computer to function as main part of the evaluation device for degree of concentration.

An evaluation device for degree of concentration according to the present invention includes: an acquirer configured to acquire a processing time measured for each of tasks performed by a user; a storage configured to store the processing time for each of the tasks acquired through the acquirer; a corrector configured to, based on load factors that are set in accordance with respective degrees of difficulty of the tasks, correct and change the processing times stored in the storage 12 to respective effective times corresponding to a prescribed degree of difficulty; and a processor configured to work out an evaluation value of user's degree of concentration based on a statistic acquired from a collection of the respective effective times for the tasks.

An evaluation device for degree of concentration according to the present invention includes: an acquirer configured to acquire a processing time measured for each of tasks performed by a user; a corrector configured to, based on load factors that are set in accordance with respective degrees of difficulty of the tasks, correct and change the processing time for each of the tasks acquired through the acquirer to an effective time corresponding to a prescribed degree of difficulty; a storage configured to store the effective times for the tasks; and a processor configured to work out an evaluation value of user's degree of concentration based on a statistic acquired from a collection of the effective times.

A program according the present invention allows a computer to function as the evaluation device for degree of concentration.

Note that a recording medium storing the program may be a computer readable medium.

With the abovementioned evaluation device and program, it is possible to work out an objective evaluation of a degree of concentration.

### BRIEF DESCRIPTION OF DRAWINGS

FIG. 1 is a block diagram illustrating an example of an embodiment;

FIG. 2 illustrates an example of a histogram of time occupancy in the embodiment; and

FIG. 3 is a block diagram illustrating another example of the embodiment.

### DESCRIPTION OF EMBODIMENTS

In the embodiment to be explained below, examples of a user include a worker in office, a learner in educational facility or learning environment, and the like. The worker in office mainly performs not physical work requiring operation results by physical movements but mental work such as documentation, information management and classification.

Hereinafter, the productivity of the mental work is called “intellectual productivity”. The intellectual productivity is influenced by not only individual knowledge and skill but also a concentration level during work (hereinafter called “a degree of concentration”), a concentration duration (hereinafter called “a concentration period”) and the like.

For example, in the case of a comparatively large mental work load, increasing the intellectual productivity requires high concentration by a mental resource for the mental work, thereby requiring a high degree of concentration. On the other hand, in the case where the mental work load is comparatively small but an amount of the work is large, the continuation of concentration by the mental resource for the mental work is required. Accordingly, the degree of concentration slightly decreases but a comparatively long concentration period is required.

The user is not limited to the worker in office, but may be a learner in school or dwelling. In the embodiment, terms such as “a mental work load”, “mental stress” and “mental” conform to the definition described in Japanese Industrial Standard (see JIS Z8502-1994).

The embodiment is configured to cause a user as an evaluation target of a degree of concentration to perform tasks. The evaluation device for degree of concentration to be explained below is configured to work out an evaluation value of a degree of concentration based on a processing time measured for each of the tasks. Here, each task means a question given to the user. The user sequentially solves the questions to be given and thereby the evaluation value of the user's degree of concentration is acquired. The evaluation device for degree of concentration then works out the evaluation value of the degree of concentration based on respective processing times for the tasks by the user.

Each task to be explained below is basically a cognitive question, and the content thereof is defined to require a solution per task of a user. A processing time is measured per task, and the device evaluation for degree of concentration works out an evaluation value of user's degree of concentration based on a processing time measured per task. Each processing time is, for example a time from the start of presentation of a corresponding task to a user to the acquisition of the solution to the task.

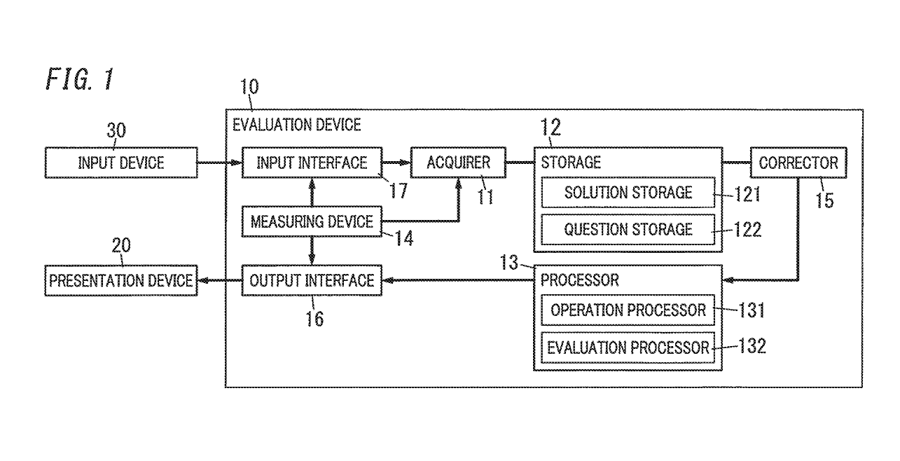

As shown in FIG. 1, the present embodiment is provided with a presentation device 20 configured to present cognitive questions, an input device 30 that allows a user to enter a solution to each cognitive question into, and an evaluation device 10 configured to work out an evaluation value of user's degree of concentration, in order to quantitatively evaluate the degree of concentration. For example, the evaluation device 10 may be combined with the presentation device 20 and the input device 30.

The evaluation device 10 may be a dedicated device, but realized by making a general purpose computer execute a program. Examples of the computer include a desktop computer, a notebook computer, a tablet computer, a smartphone, a game machine with a replaceable program, and the like. That is, in the embodiment, the computer includes a processor configured to operate according to a program, as well as a memory, an input device, an output device and the like.

In the case of the evaluation device 10 selected from the notebook computer, the tablet computer, the smartphone, the game machine and the like, it may be provided integrally with at least an input device 30, and, further provided integrally with the presentation device 20 as the output device. The embodiment has any one of a configuration in which the presentation device 20 and the input device 30 are separated from the evaluation device 10, a configuration in which the evaluation device 10 is provided integrally with the input device 30, and a configuration in which the evaluation device 10 is provided integrally with the presentation device 20 and the input device 30.

The presentation device 20 may be configured to present two or more cognitive questions simultaneously. However, the presentation device 20 is desirably configured to present the cognitive questions one by one because it can conveniently measure a time from the presentation of each cognitive question by the presentation device 20 to the entry of a corresponding solution into the input device 30, namely respective processing times for the cognitive questions. As an alternative example of the respective processing times of the cognitive questions, the presentation device 20 may be configured to display a list of the cognitive questions, regard a time till the acquisition of a first solution as a first processing time, and subsequently regard a time interval of the entry of a solution into the input device 30 as a processing time.

For example, one hundred or more cognitive questions are given to a user in order to acquire an evaluation value of user's degree of concentration. The number of the cognitive questions is not limited thereto in particular. However, the minimum number thereof may be defined so that prescribed reliability is obtained when a statistic on the respective processing times for the cognitive questions is calculated. The maximum number may be defined such that a user can continuously solve the cognitive questions.

By the way, it is generally desirable that cognitive questions for working out an evaluation value of user's degree of concentration satisfy a condition that a level of mental stress is in a specified range. However, the condition restricts the kinds of the cognitive questions satisfying. These kinds of cognitive questions often have respective different content from actual mental work. Therefore, an evaluation value of the degree of concentration obtained based on the cognitive questions may differ from the degree of concentration in the case of the actual mental work.

In the embodiment, a condition to cognitive questions is relaxed so as to allow comparatively large dispersion with respect to a level of mental stress per cognitive question. Technology proposed in the embodiment can accurately work out an evaluation value of user's degree of concentration based on respective processing times for the cognitive questions.

Incidentally, each of the cognitive questions needs to be a cognitive question by which a solution is determined uniquely. Wide kinds of cognitive questions are desirable because cognitive questions that allow practice effects to suppress the difference between characteristics of a processing time on concentration and characteristics of a processing time on non-concentration are not suitable for the calculation of an evaluation value of a degree of concentration. That is, it is desirable that the cognitive questions presented by the presentation device 20 be designed so that a difference easily occurs between characteristics of a processing time on concentration and characteristics of a processing time on non-concentration regardless of practice effects. Too low mental stress per cognitive question palls on a user, and too high mental stress increases user's tiredness. That is, too low or high mental stress per cognitive question decreases user's volition, and therefore moderate mental stress needs to be set.

As stated above, the present embodiment is configured to work out an evaluation value of a degree of concentration based on respective processing times for the cognitive questions presented by the presentation device 20. Note that each of the processing times is a processing time as a result of the entry of a correct solution into the input device 30 with respect to a corresponding cognitive question presented by the presentation device 20 because it is not possible to exclude the possibility of the entry of a solution into the input device 30 without execution of user's mental work when the user's solution entered into the input device 30 is incorrect. It is therefore desirable that when user's solution with respect; to a corresponding cognitive question is incorrect, the processing time for the cognitive question be excluded from an evaluation target. Alternatively, the evaluation device 10 may multiply an evaluation value of a degree of concentration by a coefficient that is set based on an accuracy rate of the solutions. With this configuration, if the accuracy rate is low, the evaluation value of the degree of concentration also becomes low.

The evaluation device 10 may include, as main hardware elements, a device including a processor configured to operate according to a program, and an interface device for the connection with an external device. The device including the processor may be a microprocessor or a microcomputer (a microcontroller). The interface device may have a function for the connection with at least the presentation device 20 and the input device 30. Desirably, the interface device has a function for data communication. The program to be executed by the processor may be stored in a ROM (Read Only Memory) in advance, or provided via a telecommunications network such as the Internet or by a computer readable medium.

As shown in FIG. 1, the evaluation device 10 may include an acquirer 11 configured to acquire a processing time measured for each of the cognitive questions (the tasks) performed by a user. The evaluation device 10 may also include an output interface 16 for the connection with the presentation device 20, and an input interface 17 for the connection with the input device 30. The output interface 16 is configured to allow the presentation device 20 to present the cognitive questions. The input interface 17 is configured to receive a solution entered into the input device 30.

The respective processing times for the cognitive questions may be measured by the input device 30 or by a measuring device 14 in the evaluation device 10. In the case where the input device 30 measures each processing time, the input device 30 may interact with the presentation device 20 and measure, as a processing time, a time from the presentation of a cognitive question by the presentation device 20 to the entry of a solution thereof. In the case where the measuring device 14 measures each processing time, the measuring device 14 may measure, as a processing time, a time from the output of a cognitive question through the output interface 16 to the reception of a solution thereof by the input interface 17. Therefore, the acquirer 11 in the former configuration acquires each processing time from the input device 30 via the input interface 17, while the acquirer 11 in the former configuration acquires each processing time from the measuring device 14.

The evaluation device 10 includes a storage 12 configured to store information on the respective processing times acquired with respect to the cognitive questions, and a processor 13 configured to work out an evaluation value of user's degree of concentration based on the information stored in the storage 12. The storage 12 may store the respective processing times for the cognitive questions presented to the user, in presentation order thereof. The storage 12 may also store along with respective absolute times (date and time combinations) when the cognitive questions are presented.

A solution storage 121 provided in the storage 12 is configured to store the information on a processing time acquired with the acquirer 11 per cognitive question. The solution storage 121 may store a solution result showing correct or incorrect per cognitive question. That is, when a user enters a solution into the input device 30 with a cognitive question presented by the presentation device 20, the solution storage 121 may store information on a processing time for the cognitive question and on a solution result showing correct or incorrect. If a solution is entered into the input device 30, the solution storage 121 may also store information for identifying a corresponding cognitive question. Alternately, the solution storage 121 may store respective load factors defined for the cognitive questions in advance. The load factors will be described later.

The predetermined number of cognitive questions may constitute a cognitive question set. In this case, the processor 13 may work out an accuracy rate of the cognitive question set after respective solutions of the cognitive question set are acquired. The solution storage 121 may store the accuracy rate worked out through the processor 13. Note that the solution storage 121 may store not both respective processing times for the cognitive questions and the solution results showing correct or incorrect, but only the respective processing times. The cognitive question set may be a hundred or more cognitive questions. An evaluation value of user's degree of concentration can be worked out only from the cognitive question set. In addition, a change over time in a degree of concentration can be obtained from two or more cognitive question sets.

The storage 12 may further include a question storage 122 configured to store cognitive questions to be presented through the presentation device 20. The question storage 122 may store not only the cognitive questions but also correct solutions each of which is assigned to a corresponding cognitive question. On the other hand, the processor 13 may include an evaluation processor 132 configured to select a cognitive question for one measurement from the cognitive questions stored in the question storage 122. The evaluation processor 132 may provide the output interface 16 with the selected cognitive question to allow the presentation device 20 to present the cognitive question through the output interface 16.

The processor 13 may include an operation processor 131 configured to work out an evaluation value of user's degree of concentration. After the presentation device 20 presents the cognitive question set and the solution storage 121 stores user's solutions entered into the input device 30, the operation processor 131 may work out an evaluation value of user's degree of concentration based on the information on the processing times stored in the solution storage 121. That is, the operation processor 131 may work out the evaluation value of the user's degree of concentration based on the information on the processing times corresponding to the cognitive question set.

Incidentally, the embodiment admits a comparatively large dispersion of a level of mental stress per cognitive question, and accordingly each processing time varies according to not only user's degree of concentration but also a degree of difficulty of a corresponding cognitive question. Therefore, figuring out the evaluation value of the user's degree of concentration based on the processing times requires removing respective variations caused by the degree of difficulty from the processing times.

For this purpose the evaluation device 10 may include a corrector 15 configured to correct an change the processing time acquired with the acquirer 11 to an effective time corresponding to a predetermined degree of difficulty. That is, the corrector 15 may suppress the dispersion of the processing times caused by the respective degrees of difficulty of the cognitive questions to about equalize the degrees of difficulty thereof, thereby correcting the processing times. The specific correction method of the processing times through the corrector 15 will be described, later.

Hereinafter, a process performed by the operation processor 131 is explained. The operation processor 131 works out an evaluation value of user's degree of concentration based on not the processing times but also the effective times corrected through the corrector 15. In an example to be explained below, the solution storage 121 stores the processing times, and the corrector 15 then corrects and changes the processing times stored in solution storage 121 to the effective times, thereby providing the operation processor 131 with the effective times.

An effective time measured for each of the cognitive question set may be long if an elapsed time from the start of each measurement is long. This is considered to represent a decline in vigor caused by user's mental fatigue. In order to extract a feature value of the effective times, the operation processor 131 may make frequency distribution of the effective times acquired based on the processing times stored in the solution storage 121. The frequency distribution is made from a collection of the effective times, thereby making it possible to extract the feature value of the collection of the effective times. That is, an evaluation value of user's degree of concentration can be worked out based on the collection of the effective times.

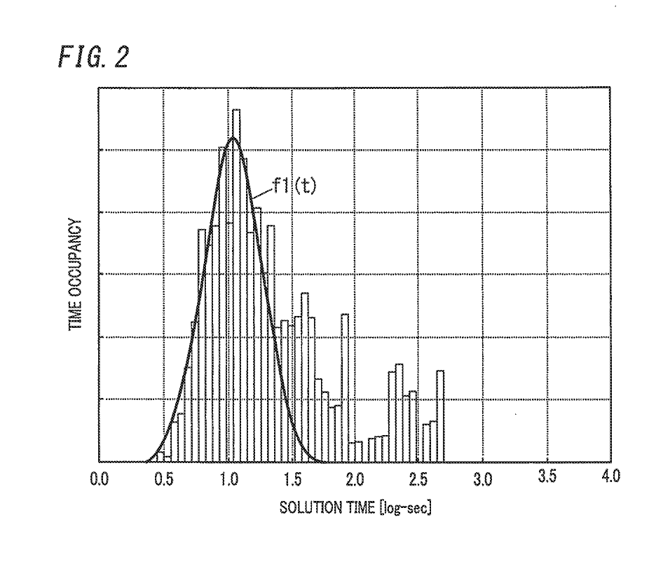

For example, a histogram of time occupancy as shown in FIG. 2 can be acquired by making the frequency distribution from the effective times acquired by the correction of the processing times stored in the solution storage 121. In FIG. 2, an effective time is described as a solution time that means a time to find a solution of each cognitive question. In the histogram of time occupancy, the effective times are divided into a plurality of segments. The histogram represents, as time occupancy, a ratio of the total of effective times per segment to the ground total of the effective times. That is, a horizontal axis with logarithmic scale represents an effective time (a solution time), and a vertical axis represents the time occupancy. The time occupancy is given by ΣTi/ΣT, where ΣT is a total of effective times in one measurement and ΣTi is a total of effective times in a segment i. In the case of appropriate cognitive questions, the experimental results show that a histogram of time occupancy has two or more peaks. That is, the histogram includes two or more regions each of which is in the shape of a mountain.

The present embodiment is configured to perform an analysis of user's degree of concentration based on the histogram of time occupancy and based on a model in which three states to occur during user's mental work are divided into a “work state”, a “short break” and a “long break”. In the “work state”, a cognitive resource is assigned to a target, and the work state shows a state that work processing advances. In the “short break”, the cognitive resource is assigned to the target, but the short break shows a state in which work processing inadvertently stops for a short time. This state will physiologically occur at a constant probability. In the “long break”, the cognitive resource is not assigned to the target, and the long break shows a state of taking a long break.

In the abovementioned “work state” and “short break”, since the cognitive resource is assigned to the target, each of them is considered to be in a state of concentration. In the “long break”, since the cognitive resource is not assigned to the target, it is considered not to be in a state of concentration. A region in the shape of a mountain including a peak, an effective time of which is shortest, is estimated to be a state in which the “work state” and the “short break” mix. A region in the shape of a mountain including any of other peaks can be interpreted as a state in which the “work state”, the “short break” and the “long break” mix because even if it is in a state of concentration, an effective time thereof may be long owing to dispersion in respective degrees of difficulty of the questions.

Incidentally, there is a knowledge on the region in the shape of a mountain, in which the “work state” and the “short break” mix. According to this knowledge, the region can be approximately applied to a probability density function of logarithmic normal distribution. On the other hand, there are some results by which the region in the shape of a mountain, in which the “work state”, the “short break” and the “long break” mix, may not be applied to the logarithmic normal distribution because the region depends on differences among individuals. The results are considered to occur depending on differences among individuals with respect to the “long break”.

When a user solves the cognitive questions in order to make a histogram of time occupancy, the user tries to keep the state of concentration. It is therefore estimated that an effective time, time occupancy of which is maximum, reflects a state in which the “work state” and the “short break” mix. Therefore, in an example of an ideal state in which respective degrees of difficulty of the questions are constant, it is estimated that the region in the shape of a mountain, in which an effective time in the histogram of time occupancy is shortest, can be approximated by the probability density function of logarithmic normal distribution, f1(t) as a function of an effective time t. However, the dispersion in the respective degrees of difficulty of the questions cannot be completely removed in reality. Therefore, in a region in the shape of a mountain, having the shortest effective time of two regions in the shape of a mountain, it is estimated that only a section having an effective time shorter than that of a peak and a section around the peak coincide with the probability density function of logarithmic normal distribution f1(t). Parameters (an expected value and variance) are determined so as to approximate the sections.

The expected value of the effective times can be calculated if the parameters of the probability density function of logarithmic normal distribution f1(t) are determined. It can be estimated that a result obtained by multiplying the calculated expected value and the total number of the questions is a time in a state of concentration, of all effective times from start to finish of each question by a user. It can be also estimated that a time obtained by subtracting the time in the state of concentration from a sum of the respective effective times is a time in a state of non-concentration. Therefore, a ratio of the time in the state of concentration to the sum of the respective effective times is defined as a concentration time ratio, and it is judged that a degree of concentration having a higher concentration time ratio is higher degree of concentration. The concentration time ratio acquired like this becomes an evaluation value of a degree of concentration.

In order to work out an evaluation value of a degree of concentration (a concentration time ratio) as stated above, the operation processor 131 may include a function for making a histogram of time occupancy, and a function for applying a function to the histogram of time occupancy. The operation processor 131 may further include a function for working out a concentration time or an evaluation value of a degree of concentration based on the parameters of the applied function.

The operation processor 131 is configured to allow the presentation device 20 to present the abovementioned concentration time ratio as the evaluation value of the degree of concentration. That is, the operation processor 131 may work out the concentration time ratio to allow the presentation device 20 to present it through the output interface 16.

Incidentally, the corrector 15 may have a function for working out a load factor in accordance with a degree of difficulty of a given cognitive question to correct and change a corresponding processing time to an effective time based on the load factor. In this example, it is estimated that the degree of difficulty of the cognitive question is proportional to the processing time under the condition that user's degrees of concentration are equal to each other. It is also estimated that respective degree of difficulty of the cognitive questions corresponds to respective user's mental stress if the differences in respective ability among users are not considered. In such a relation, effective times from which the degrees of difficulty of the cognitive questions are removed can be calculated by multiplying each processing times for the cognitive questions by a load factor, where the load factor is a reciprocal number of a corresponding degree of difficulty.

Hereinafter, a calculation example of the effective times is explained. In this example, the degree of difficulty when the processing time is 4 seconds is 1, and the degree of difficulty is defined as a standard value. In this example, if a processing time for a given cognitive question is 6 seconds, the degree of difficulty of the cognitive question is 1.5 based on the abovementioned definition. An effective time of the cognitive question when the processing time is 6 seconds is 6 seconds×(1/1.5)=4 seconds based the reciprocal degree of difficulty. Thus, effective times free from respective degree of difficulty of the cognitive questions can be worked out.

Since the abovementioned load factor is defined based on the condition that user's degrees of concentration are equal to each other, the load factor needs to be determined so that the condition is satisfied. Therefore, in the embodiment, a load factor per cognitive question is defined based on any of three kinds of methods below. That is, the load factor is selected from: (1) a theoretical value obtained by analyzing a process of the cognitive question; (2) an expected value obtained from evaluation experiment results; and (3) a measured value obtained as a result of user's actual solution to the cognitive question. Based on the methods for acquiring the load factor, a load factor below is defined to any of the theoretical value, the expected value and the measured value.

(1) Theoretical Value

The theoretical value of a load factor is defined by analyzing user's process of a given cognitive question. As an example, one digit number and one digit number are added, or two digit number and one digit number are added. However, rounding up is not considered in the case where two digit number and one digit number are added. As respective specific examples, the former can be shown by 3+6, and the latter can be shown by 23+6.

A process of the cognitive question of “3+6” can be divided as shown in Table 1, for example.

TABLE 1

| Step | Cognitive question |
|---|---|
| 1 | Locate a first number “3”. |
| 2 | Store the first number “3” |
| 3 | Locate operation symbol “+”. |
| 4 | Recognize operation rule. |
| 5 | Locate a second number “6”. |
| 6 | Store the second number “6”. |
| 7 | Add the second number to the first number. |
| 8 | Store the operation result. |
| 9 | Enter the solution. |

On the other hand, a process of the cognitive question of “23+6” can be divided as shown in Table 2, for example.

TABLE 2

| Step | Cognitive question |
|---|---|
| 1 | Locate a first number of “23”. |
| 2 | Store the first number of “23”. |
| 3 | Locate operation symbol “+”. |
| 4 | Recognize operation rule. |
| 5 | Locate a second number of “6”. |
| 6 | Store the second number of “6”. |
| 7 | Add the second number to the one's digit of the first number. |
| 8 | Store the operation result. |
| 9 | Calculate the sum of the operation result and the ten's digit |
| | of the first number. |
| 10 | Store the operation result. |
| 11 | Enter the solution. |

If each process of the cognitive questions is divided as shown in Table 1 or 2, the cognitive question of “3+6” has 9 steps, while the cognitive question of “23+6” has 11 steps. When a ratio of the number of steps is regarded as a ratio of a degree of difficulty, the degree of difficulty of the cognitive question of “23+6” to the cognitive question of “3+6” is given by 11/9≈1.2. In the case of this example, based on a standard that is a degree of difficulty of a cognitive question in which both one's digits are added, a theoretical value of the load factor is given by 1/1.2≈0.82.

The method for dividing respective processes of the cognitive questions is not limited to the examples of Tables 1 and 2, but other processes may be applied. A level of mental stress may be set to individual cognitive questions in a process and a sum of levels of mental stress may be replaced with the number of steps. In an example, each level of respective mental stress from the first step to the eighth step in Table 1 is 1.0, and a level of mental stress at the ninth step is 1.1. In this case, the number of steps about “3+6” is 9.1. Thus, respective load factors for the cognitive questions can be worked out more accurately based on a level of mental stress per step.

(2) Expected Value

An expected value of a load factor can be worked out by providing subjects with a cognitive question with a degree of difficulty as a standard value and a cognitive question as a comparison target to work out respective degrees of difficulty of the cognitive questions based on respective processing times for the cognitive questions. The conditions other than the cognitive questions are equal to each other among the subjects. A ratio of a processing time for the cognitive question having the degree of difficulty as the standard value and a processing time for the specified cognitive question as the comparison target is worked out for each of the plurality of subjects. A mean value of the ratios worked out as described above is an expected value of a load factor for the cognitive question as the comparison target.

In an example, the processing time for the cognitive question having the degree of difficulty as the standard value is T1, and the processing time for the cognitive question as the comparison target is Tx. A ratio of a processing time corresponding to a load factor per subject is given by Tx/T1, and a mean value of respective ratios Tx/T1 among the plurality of subjects can be worked out as an expected value of a load factor for a corresponding cognitive question.

If processing conditions for cognitive questions are equal to each other among the subjects, subject's degree of concentration is not influenced by circumstances. Therefore, the processing time is influenced only by a degree of difficulty of a corresponding cognitive question and subject's processing capacity for the cognitive question. Since the ratio Tx/T1 is worked out per subject, the influence by the subject's processing capacity can be removed. In addition, the ratio Tx/T1 is worked out per subject, thereby reducing differences in user interfaces such as the presentation device 20 and the input device 30 and differences in respective subject's motivation.

The method for working out the expected value has the highest generalized method of the three kinds of methods for working out the load factor. In particular, the cognitive questions may be any of questions about calculation, questions about language, question about comparison and judgment, and questions to be solved based on regulations. It is accordingly possible to work out an expected value of a load factor for each of the cognitive questions even in the case where the questions mix. A load factor can be also worked out per cognitive question even in the case where circumstances for processing of the cognitive questions are different from each other among subjects because an expected value of the load factor is worked out per subject based on the ratio Tx/T1.

(3) Measured Value

The theoretical value and the expected value of the load factor are worked out before a user solves the cognitive questions in order to work out an evaluation value of a degree of concentration. On the other hand, the measured value of the load factor is worked out in a period of time when a user solves the cognitive questions in order to work out an evaluation value of user's degree of concentration.

The user is provided with cognitive questions having the degree of difficulty as the standard value and cognitive questions as the comparison targets, like the case where the expected value of the load factor is worked out. Respective degrees of difficulty of the cognitive questions are unknown, but respective degrees of difficulty of the cognitive questions to be presented in the same format are similar to each other. It is therefore possible to acquire a standard of a processing time in accordance with the degree of difficulty by working out a mean value of processing times, respective degrees of difficulty of which are similar to each other. A ratio therefore corresponds to the load factor, where the ratio is obtained from a mean value of processing times for cognitive questions having similar degrees of difficulty within the cognitive questions as the comparison target, and a mean value of processing times for cognitive questions having a degree of difficulty as the standard value.

In short, the expected value of the load factor can be worked out by providing subjects with the same cognitive questions, whereas the measured value of the load factor can be worked, out by providing a user with the cognitive questions having similar degrees of difficulty. The “subject” solves the cognitive questions in order to acquire the expected value in consideration of the case where a user solving the cognitive questions in order to acquire the load factor often differs from a user measuring the degree of concentration. On the other hand, the “user” solves the cognitive questions in order to work out the measured value in consideration of the case where a user solving the cognitive questions in order to acquire the load factor coincides with a user measuring the degree of concentration.

Even in the case of similar degrees of difficulty, a change occurs in a degree of concentration, and it is therefore desirable that each cognitive question having the degree of difficulty as the standard value and each cognitive question as the comparison target having a similar degree of difficulty are provided continuously. In order to further reduce the influence of a change in a degree of concentration on the processing time, the load factor may be worked out by exchanging the order of the cognitive question as the comparison target and the cognitive question having the degree of difficulty as the standard value at appropriate timing to define a mean value of the load factors obtained by the exchange of the cognitive questions as the load factor.

The evaluation of respective degrees of difficulty of the cognitive questions can be defined in advance based on the similarity in types of the cognitive questions. The respective degrees of difficulty may be judged whether to be similar based on subject's processing times measured for each of the cognitive questions.

The measured value of the load factor obtained as described above represents a value reflecting a degree of difficulty of a corresponding cognitive question as long as the cognitive question is provided to a user at appropriate timing. In particular, the load factor is obtained from a ratio of a cognitive question as the comparison target and a cognitive question having a degree of difficulty as a standard value. It is according possible to reduce differences in the user interfaces such as the presentation device 20 and the input device 30, and differences in respective subject's motivation. The influence by subject's processing capacity can be removed by the same reason.

The cognitive questions may be any of questions about calculation, questions about language, question about comparison and judgment, and questions to be solved based on regulations. Even if they mix, each measured value of respective load factors of the cognitive questions can be worked out.

As stated above, if a load factor is worked out per cognitive question, it is possible to correct and change the measured processing time to a processing time corresponding to a prescribed degree of difficulty by applying a load factor in accordance with, the cognitive question to the processing time measured with respect to the cognitive question. Hereinafter, an application example of the evaluation device 10 when an evaluation value of user's degree of concentration is worked out in actuality will be explained.

Examples of cognitive questions which are similar to each other and have respective different degrees of difficulty include addition of one digit number to one digit number without rounding up, addition of one digit number to one digit number with rounding up, and subtraction of one digit number from one digit number. In the case where cognitive questions contain three kinds of cognitive questions, it is possible to assign a load factor per kind. A representative value of processing times may be worked out for each kind of the cognitive questions in order to work out a load factor for each kind of the cognitive questions. The representative value may be a mean value or a median value.

In an example, a processing time for a cognitive question about the addition of one digit number to one digit number without rounding up is a standard value, and the other cognitive questions are comparison targets. Desirably, load factors are further divided for each of age groups because processing times may vary according to user's age. There is also a possibility that processing times vary according to body condition, habit and the like besides the age groups, and therefore these factors may be considered additionally.

As stated above, it is considered that an evaluation value of a degree of concentration varies according to user characteristics such as user's age, body condition, habit and the like, as well as usage environment of the evaluation device 10. Examples of the usage environment include illuminance, temperature, time range as well as a presentation method of the cognitive questions. Examples of the presentation method of the cognitive questions include a method for providing similar cognitive questions, a method for providing different kinds of cognitive questions, a method for providing cognitive questions during another behavior such as a game.

It is therefore necessary to not only select a cognitive question to be given to a user but also set user's characteristics and usage environment when the evaluation device 10 works out an evaluation value of user's degree of concentration. In the embodiment, each load factor of the cognitive questions is defined as a theoretical value or an expected value in advance. In this case, the load factors of the cognitive questions may be stored for each of the cognitive questions in the question storage 122.

In the case where each load factor is defined as a measured value, the corrector 15 may calculate each load factor based on a processing time per cognitive question while providing a user with the cognitive questions for working out an evaluation value of a degree of concentration. It is desirable that user's characteristics and usage environment are equal among different users when a measured value is worked out for each of the load factors of the cognitive questions. The load factors are calculated by the method as stated above.

As stated above, the load factors, namely the respective degrees of difficulty of the tasks can be defined based on the number of steps in a cognitive process per task. Alternatively, each degree of difficulty may be defined based on a ratio of a processing time for a task as a comparative target to a processing time for a task as standard.

The evaluation device 10 is configured to acquire respective processing times for the cognitive questions to store, in the solution storage 121, the respective processing times for the cognitive questions and respective solution results showing correct or incorrect of the cognitive questions, in order to work out an evaluation value of user's degree of concentration. The solution storage 121 may store information for identifying the cognitive questions along with information on the respective processing times and the respective solution results showing correct or incorrect.

After the solution storage 121 stores a requisite number of processing times for working out an evaluation value of user's degree of concentration, the corrector 15 reads a processing time sequentially from the solution storage 121. The corrector 15 extracts a load factor of a corresponding cognitive question by checking the information for identifying the cognitive question with the information of the question storage 122. The extracted load factor is applied to the processing time read from the solution storage 121, and the corrector 15 corrects and changes it to a processing time corresponding to a prescribed degree of difficulty.

Effective times as a result of the correction of the processing times by the corrector 15 are given to the operation processor 131. The frequency distribution of the effective times is obtained as stated above, and the degrees of concentration are worked out quantitatively based on the frequency distribution.

In the abovementioned configuration example, only the cognitive questions are presented to a user, but if a load factors per cognitive question is worked out, the cognitive questions, the respective load factors of which are worked out may be contained in a different work. The different work is not limited to a work associated with learning or business. For example, the cognitive questions may be contained in the content of an electronic amusement game.

Examples of a device for the electronic game include a game machine, and a general purpose device such as a smartphone, a tablet computer, and a personal computer. Any of the devices includes a processor configured to execute a program, and devices functioning as the presentation device 20 and the input device 30. The processor executes the program, thereby realizing the abovementioned function of the evaluation device 10.

The program may be provided by a computer readable medium, or via a telecommunications network such as the Internet. In the case of a dedicated device only for a specified electronic game, the program may be stored in a ROM (Read Only Memory) in advance.

In timing for presenting cognitive questions in an electronic game, it is desirable that after response to respective similar content in the electronic game, similar cognitive questions be presented. The respective similar content requires respective similar operations. The similar cognitive questions have an identical process. For example, respective operations of content for movement operation and content for selection operation are dealt as different operations. Desirably, the cognitive questions are presented under similar conditions.

In the embodiment, since load factors are set for respective cognitive questions, the degrees of difficulty in the cognitive questions may not be related to respective content of the electronic game. It is however desirable that the load factors of the cognitive questions be similar to the respective similar content in the electronic game. Desirably, the cognitive questions with respective similar load factors are included in a period of time in which user's degrees of concentration are similar to each other. The similar load factors mean that variance of the load factors of the cognitive questions is in a specified range.

If a user plays the electronic game, the evaluation device 10 represents a cognitive question at appropriate timing while the electronic game is played, and the storage 12 stores respective processing times for the presented cognitive questions. If the electronic game is finished or the number of the cognitive questions reaches a predetermined number (e.g., 100), the corrector 15 corrects and change the processing times stored in the storage 12 to effective times, which are given to the processor 13. The processor 13 works out degrees of concentration based on the effective times, and allows the presentation device 20 to present a result thereof through the output interface 16. The presentation device 20 may present the result at appropriate timing to be selected. The output interface 16 may present the result automatically at the end of the electronic game, or present the result in accordance with an instruction entered into the input device 30 by a user.

In the abovementioned configuration example, the presentation device 20 may double as a display for displaying the content of the electronic game. The input device 30 may also double as a keyboard or a touch panel for the operation of the electronic game. In other words, the game machine for the electronic game may include an application program for realizing the evaluation device 10 explained in the embodiment. In this case, it is desirable that the application program for realizing the evaluation device 10 be contained in an application program for the game machine. The game machine may have dedicated hardware constituting the evaluation device 10 of the embodiment. In this case, the cognitive questions may be contained in the content of the electronic game.

Incidentally, the abovementioned electronic game is, for example a game for mainly mental work, such as a role-playing game, a simulation game, or an educational game. On the other hand, there are electronic games without mainly requiring mental work, which require body motion with respect to sensory stimulation of visual sense and auditory sense, such as games for simulating exercise. In this sort of electronic game, each processing time may be a time from the provision of sensory stimulation to a specified body motion.

In this sort of electronic game, there is a possibility that the interest in the game may be spoiled as a result of the inclusion of cognitive questions requiring mental work in the content thereof. Accordingly, each processing time is defined by a time from the sensory stimulation to a processing operation by user's body. It is also desirable that each processing time be corrected based on an appropriate weighting factor according to correct or incorrect motion because a user may perform wrong motion with respect to a request of body's specified motion. For example, the load factor for the processing time by the wrong motion may be relatively increased to approach a non-concentration state when an evaluation value of a degree of concentration is worked out, thereby correcting the processing time so as to make it longer than the measured value.

In an example in which cognitive questions are contained in the content of the electronic game, a load factor per cognitive question is defined in advance based on the theoretical value or the expected value as stated above. On the other hand, in the case where respective measured values of the load factors are acquired with respect to a user, the evaluation device 10 acquires the measured values by presenting the cognitive questions to a user like the case where respective theoretical values of the load factors are worked out as stated above.

The processing times are stored in the storage 12, and the processor 13 works out a load factor per cognitive question based on the processing times. The corrector 15 then applies the processing times stored in the storage 12 to respective load factors, thereby working out effective times to provide the processor 13 with the effective times. The processor 13 works out an evaluation value of a degree of concentration based on the effective times to allow the presentation device 20 to present the evaluation value via the output interface 16. Note that a period of time to present cognitive questions in order to work out the load factors may be different from a period time to present cognitive questions in order to work out an evaluation value of a degree of concentration.

The abovementioned cognitive questions are cognitive questions having one kind of process in any case. For example, different kinds of processes are selectable with respect to the content of the electronic game in general, and therefore the content of the electronic game or the like is unsuitable for cognitive questions for working out an evaluation value of a degree of concentration. In short, the cognitive questions need to be set so that respective solutions are acquired uniquely.

The tasks for working out an evaluation value of a degree of concentration may not necessarily be cognitive questions. That is, even if they are not the cognitive questions, it is possible to work out an evaluation value of a degree of concentration objectively and quantitatively as long as respective processing times for the tasks can be measured. As stated above, the tasks for measuring the processing times may be mixed with works that differ from the tasks for measuring the processing times. The user therefore deals with the tasks for measuring the processing times while performing any work, and it is thereby possible to evaluate a change in user's degree of concentration in operation.

In the case where a physical event occurs and one task is repeated while a user deals with the task, if a processing time for the task can be measured, an evaluation value of a degree of concentration can be worked out based on the processing time. For example, when vegetable is cut into fine strips with a kitchen knife, it is possible to measure a processing time, where one cutting of the vegetable is the processing of the task, a sound occurring whenever the task is dealt with is the physical event, and the processing time is a requisite time for the one cutting of the vegetable. That is, if measuring processing times each of which is a time interval between collision noises occurring whenever the kitchen knife collides with a chopping board during the cutting, the processor 13 can work out an evaluation value of a degree of concentration by a user performing the cutting in accordance with the abovementioned procedure based on the processing times. For example, each processing time may be measured by capturing an image of the processing by a camera or the like.

The presentation device 20 may be separated from the evaluation device 10. In this case, the presentation device 20 may be replaced with an electronic paper or a paper. When the presentation device 20 is replaced with the electronic paper or the paper, a time interval whenever a solution is entered into the input device 30 may be regarded as a processing time for a corresponding cognitive question.

When the presentation device 20 is replaced with the paper, individual identification information such as a two-dimensional code may be additionally written per cognitive question, in order to identify the presented cognitive question. In this case, desirably the identification information may be read by a reader such as a camera. With this configuration, it is possible to recognize tasks dealt with by a user to assign respective processing times to the tasks, based on respective identification information read by the reader. Alternatively, in the input device 30, solution entry fields (solution entry boxes) may be assigned to respective tasks (cognitive questions), and it is thereby possible to assign respective processing times to the tasks.

When the presentation device 20 operates along with an electronic paper, the presentation device 20 may be provided with a function for communicating with another device. In this configuration, the another device provides the presentation device 20 with information for presenting cognitive questions, and provides the evaluation device 10 with respective times corresponding to the information, thereby enabling accurate measurement of a processing time per cognitive question.

The abovementioned embodiment as an example is configured to allow the presentation device 20 to present cognitive questions while sequentially presenting the cognitive questions one by one. However, the processing times may be measured based on a time between solutions. For example, a processing time per cognitive question may be a time between the end of the entry of a solution into the input device 30 to the end of the entry of a next solution. When the processing time is the time between solutions, the processing times can be worked out without the presentation device 20. For example, the processing times can be worked out by presenting a paper printed with a cognitive question(s) to a user, allowing the user to enter a solution into the input device 30, and storing a time at the end of the entry of a solution per cognitive question. When a paper printed with a cognitive question(s) is presented to a user, each paper may be printed with one cognitive question, and it is unnecessary that a list of cognitive questions is printed on one paper.

As stated above, the evaluation device 10 works out an evaluation value of user's degree of concentration to present the evaluation value through the presentation device 20. According a supporter or the like can utilize the evaluation value for user's educational support or business support. In addition, in the case where a user who is interested in a level of its own degree of concentration and performs training for improving its own concentration power, the user can know a training effect by confirming an evaluation value of the degree of concentration.

As explained above, the evaluation device for degree of concentration 10 of the embodiment includes the acquirer 11, the storage 12, the processor 13 and the corrector 15. The acquirer 11 is configured to acquire a processing time measured for each of tasks performed by a user. The storage 12 is configured to store the processing time for each of the tasks acquired through the acquirer 11. The corrector 15 is configured to, based on load factors that are set in accordance with respective degrees of difficulty of the tasks, correct and change the processing times stored in the storage 12 to respective effective times corresponding to a prescribed degree of difficulty. The processor 13 is configured to work out an evaluation value of user's degree of concentration based on a statistic acquired from a collection of the respective effective times for the tasks.

In the evaluation device for degree of concentration 10 of the embodiment, the corrector 15 may be configured to correct and change the processing times for the tasks acquired through the acquirer 11 to the respective effective times corresponding to the prescribed degree of difficulty based on the load factors that are set in accordance with respective degrees of difficulty of the tasks. In this configuration, the storage 12 may store the respective effective times for the tasks. The processor 13 may work out the evaluation value of user's degree of concentration based on the statistic acquired from the collection of the respective effective times.

With the configuration of the embodiment, the evaluation device 10 corrects and change the respective processing times for the tasks performed by the user to the respective effective times based on the respective load factors that are set in accordance with the respective degrees of difficulty of the tasks, and works out an evaluation value of a degree of concentration based on the effective times. Thus, the evaluation device 10 can work out an objective evaluation value of a degree of concentration. Moreover, the respective processing times are corrected and changed to the respective effective times by applying the load factors in accordance with the respective degrees of difficulty of the tasks. It is accordingly possible to accurately work out an evaluation value of a degree of concentration even if the tasks include dispersion in degree of difficulty.

With the configuration of the embodiment, the load factors in accordance with respective degrees of difficulty of the tasks are applied to the respective processing times for the tasks. Accordingly, the respective processing times are corrected and changed to the respective effective times in accordance with the prescribed degree of difficulty. As a result, the limitation on the tasks for working out the evaluation value of the degree of concentration can be reduced. That is, the constraint conditions for the tasks can be reduced, and therefore a burden on choice of the tasks can be reduced.

It is desirable that each of the abovementioned tasks be a work that requires a solution or a response to a given cognitive question. With this configuration, respective processing times for cognitive questions are acquired from respective response times of respective solutions or responses thereto, and an evaluation value of user's degree of concentration is worked out from the processing times. It is therefore possible to accurately work out an evaluation value of a degree of concentration in the case of mental work.

The evaluation device for degree of concentration 10 of the a embodiment may further include the output interface 16, the input interface 17 and the measuring device 14. The output interface 16 is configured to allow the presentation device 20 to present the cognitive questions. The input interface 17 is configured to receive the solution entered into the input device 30. The measuring device 14 is configured to measure a processing time that a time from presentation of a cognitive question through the presentation device 20 to entry of a solution into the input device 30. In this configuration, the acquirer 11 is configured to acquire the processing time from the measuring device 14.

With this configuration, the evaluation device 10 presents the cognitive questions and measures respective processing times, and therefore can be simply configured because an additional configuration for the presentation of the cognitive questions and the measurement of the processing times are not required.

A program of the embodiment is a program for allowing a computer to function as the abovementioned evaluation device 10. This program may be stored in a computer readable medium. In this case, the computer can execute the program stored in the medium, or after the program stored in the medium is installed in the computer, the computer can execute the program. The program may be provided via a telecommunications network such as the Internet. An objective evaluation value of a degree of concentration can be worked out through the program.

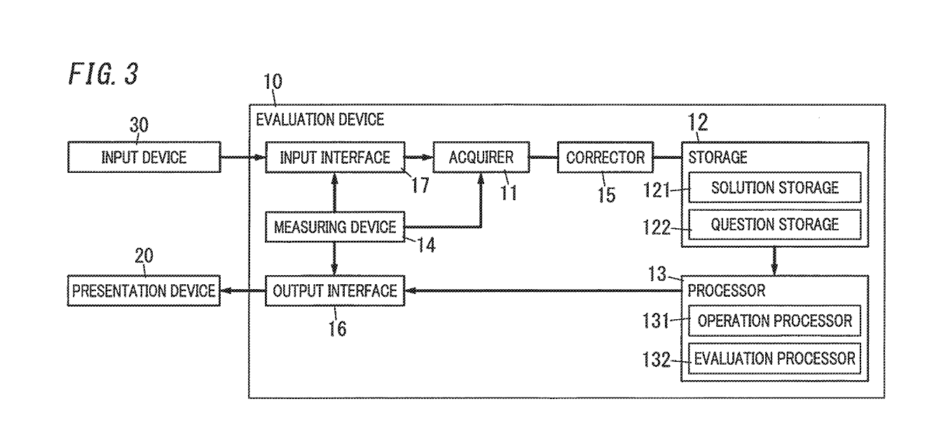

With the abovementioned configuration example, the storage 12 stores the processing times acquired through the acquirer 11, and the corrector 15 corrects and changes the processing times stored in the storage 12 to respective effective times to provide the effective times to the processor 13. In contrast, as shown in FIG. 3, the corrector 15 may correct and change a processing time acquired through the acquirer 11 to an effective times to store the effective time in the storage 12. In this configuration, the processor 13 may work out an evaluation value of user's degree of concentration based on the effective times stored in the storage 12.

The abovementioned embodiment is an example of the invention. The invention is therefore not limited to the abovementioned embodiment. Numerous modifications other than this embodiment can be made in accordance with design or the like without departing from the true scope of the present invention.

1. An evaluation device for degree of concentration, comprising:
an acquirer configured to acquire a processing time measured for each of tasks performed by a user;
a storage configured to store the processing time for each of the tasks acquired through the acquirer;
a corrector configured to, based on load factors that are set in accordance with respective degrees of difficulty of the tasks, correct and change the processing times stored in the storage to respective effective times corresponding to a prescribed degree of difficulty; and
a processor configured to work out an evaluation value of user's degree of concentration based on a statistic acquired from a collection of the respective effective times for the tasks.

2. An evaluation device for degree of concentration, comprising:
an acquirer configured to acquire a processing time measured for each of tasks performed by a user;
a corrector configured to, based on load factors that are set in accordance with respective degrees of difficulty of the tasks, correct and change the processing time for each of the tasks acquired through the acquirer to an effective time corresponding to a prescribed degree of difficulty;
a storage configured to store the effective times for the tasks; and
a processor configured to work out an evaluation value of user's degree of concentration based on a statistic acquired from a collection of the effective times.

3. The evaluation device for degree of concentration, of claim 1, wherein each of the tasks is a work that requires a solution or a response to a given cognitive question.

4. The evaluation device for degree of concentration, of claim 3, further comprising:
an output interface configured to allow a presentation device to present the cognitive questions;
an input interface configured to receive a solution entered into the input device; and
a measuring device configured to measure a processing time that a time from presentation of a cognitive question through the presentation device to entry of the solution into the input device; wherein
the acquirer is configured to acquire the processing time from the measuring device.

5. The evaluation device for degree of concentration, of claim 1, wherein each of the degrees of difficulty is defined based on a number of steps of a cognitive process acquired from a corresponding task.

6. The evaluation device for degree of concentration, of claim 1, wherein each of the degrees of difficulty is defined based on a ratio a requisite processing time for a task as a comparison target to a requisite processing time for a task as a standard.

7. A program, for allowing a computer to function as an evaluation device for degree of concentration, of claim 1.

8. The evaluation device for degree of concentration, of claim 2, wherein each of the tasks is a work that requires a solution or a response to a given cognitive question.

9. The evaluation device for degree of concentration, of claim 8, further comprising:
an output interface configured to allow a presentation device to present the cognitive questions;
an input interface configured to receive a solution entered into the input device; and
a measuring device configured to measure a processing time that a time from presentation of a cognitive question through the presentation device to entry of the solution into the input device; wherein
the acquirer is configured to acquire the processing time from the measuring device.

10. The evaluation device for degree of concentration, of claim 2, wherein each of the degrees of difficulty is defined based on a number of steps of a cognitive process acquired from a corresponding task.

11. The evaluation device for degree of concentration, of claim 2, wherein each of the degrees of difficulty is defined based on a ratio a requisite processing time for a task as a comparison target to a requisite processing time for a task as a standard.

12. A program, for allowing a computer to function as an evaluation device for degree of concentration, of claim 2.
